(19) United States
(12) Patent Application Publication
(10) Pub. No.: US 2017/0189235 A1
(43) Pub. Date: Jul. 6, 2017

(54) COMPRESSION BANDAGE FOR TREATING LYMPHATIC EDEMAS

(71) Applicant: Julius Zorn GmbH, Aichach (DE)

(72) Inventor: Uwe SCHETTLER, Aichach (DE)

(73) Assignee: Julius Zorn GmbH, Aichach (DE)

(21) Appl. No.: 15/388,257

(22) Filed: Dec. 22, 2016

(30) Foreign Application Priority Data
Dec. 30, 2015 (DE) 20 2015 107 145.0

(51) Int. Cl. A61F 13/00; A61F 13/08

(52) U.S. Cl. CPC A61F 13/00038; A61F 13/00029; A61F 13/085; A61F 2013/0028; A61F 2013/00093

(57) ABSTRACT

A compression bandage for treating lymphatic edema. The compression bandage includes a bandage body made of a resilient material to be wrapped around the body extremity, with a first group of fastening straps. The fastening straps of the first group are arranged on an end section of the bandage body, are separated from one another by notches in the bandage body, and, in each case, have first fastening elements, by which the respective fastening strap of the first group can be fastened to the outside of the bandage body when the body bandage is wrapped around the body extremity. In order to prevent the swollen body tissue from being able to pass through, between the notches in the first group of fastening straps, a second group of fastening straps is provided, which is fastened to the outside of the bandage body and whose fastening straps are arranged so that they cover the notches when the compression bandage is applied on a body extremity.

### FIELD OF THE DISCLOSURE

The disclosure relates to a compression bandage for treating lymphatic edemas.

### BACKGROUND

Lymphatic edemas are fluid accumulations in the intercellular space that are caused by mechanical insufficiency of the lymphatic vessel system of the human or animal body. The cause can be a chronic inflammatory medical condition specifically affecting the body extremities. Due to the mechanical insufficiency, the body fluid in the intercellular space can no longer be removed sufficiently through the lymph vessels, which leads to blockage and accumulation of fluid in the intercellular spaces.

From the prior art, it is known how to treat lymphatic edemas by compression of the affected body extremities. For this purpose, for example, compression bandages are wrapped around the affected arms or legs in order to exert an external compression pressure. The compression pressure promotes the removal of lymph fluid and as a result the degradation of the lymph blockage.

From EP 1 735 019B1, a compression bandage for treating lymphatic edemas is known, which comprises a first and a second band, each wrapped around a section of the body extremity to be treated and secured by means of fastening elements in such a manner that they exert predetermined compression pressure on the body extremity. Here, the two bands are attached next to one another and they comprise short-stretch bandages that have a maximum elastomer stretching range from 15% to 100% of an unstretched state, and exert a compression pressure in predetermined pressure ranges on the body extremity, when they are wrapped at the body extremity at maximum stretching around the extremity. The desired amount of compression of a band is obtained here by varying the length and width of the band and/or of the composition of the band. The short-stretch bandages here comprise a support material and an elastomer material.

Another compression bandage for treating lymphedemas is known from U.S. Pat. No. 7,329,232 B2, wherein this compression bandage as well comprises several bands extending from a flat central area, which can be wrapped around a body extremity to be treated and fastened by fastening elements to the flat central area of the bandage in order to exert a compression pressure on the body extremity when the bandage is applied.

In the known compression bandages, a desired compression pressure can be generated by differential stretching of the bands and by fastening the bands in a certain position. Since each of the bands has a stretching range, predetermined by the material of the band, with a maximum stretching in comparison to the unstretched state, the achievable range of the compression pressure, however, has to be predetermined and limited by the stretching characteristic of the individual bands.

In the known compression bandages, it can moreover occur that the body tissue of the extremity, which is swollen due to the lymphatic insufficiency, can squeeze out between adjacent bands that are wound around different sections of the body extremity, which can lead to a diminished therapeutic effect of the compression bandage.

### SUMMARY

On this basis, the disclosure provides a compression bandage for treating lymphatic edemas, by means of which a broad range of compression pressures can be achieved and the squeezing out of swollen body tissue of the body extremity to be treated can be largely prevented.

Preferred embodiment examples of the compression bandage according to the disclosure are also presented.

The compression bandage comprises a bandage body made of a resilient material to be wrapped around the body extremity to be treated, and having an inner side and an outside as well as a first and second end section of the bandage body, as well as a first group of fastening straps and a second group of fastening straps, wherein the first group of fastening straps is arranged at an end section of the bandage body and comprises a plurality of fastening straps that advantageously abut against one another, are separated from one another by notches in the bandage body, and, in each case, have first fastening means, by means of which the respective fastening strap of the first group of fastening straps can be secured to the outside of the bandage body. The second group of fastening straps here also comprises a plurality of fastening straps, which advantageously abut against one another, are separated by notches, and can be fastened to the outside of the bandage body in such a manner that the notches between the fastening straps of the first group overlap when the compression bandage is applied against the body extremity.

With this compression bandage according to the disclosure, it is possible, due to the first group of fastening straps and the second group of fastening straps, to vary the compression pressure exerted by the compression bandage on the body extremity in broad ranges, in that, first, by means of the first fastening straps, a predetermined compression pressure is generated, and said compression pressure is then varied further by means of the fastening straps of the second group, in that, with the fastening straps of the first group, an additional stretching of the bandage body and thus an even higher compression on the body extremity is generated. The fastening straps of the first group and of the second group here are fastened in each case via first or second fastening means, when the bandage bodies are wrapped around the body extremity, onto the outside of the bandage body. When the compression bandage is applied, the fastening straps of the second group cover the fastening straps of the first group in the area of the notches, which separate the fastening straps of the first group from one another. This prevents the swollen body tissue from being able to squeeze through, between adjacent fastening straps of the first group. Preferably, the fastening straps of the second group are arranged offset in the longitudinal direction of the bandage body with respect to the first fastening straps, so that the notches between adjacent fastening straps of the first group are covered in each case by one of the fastening straps of the second group. In this way, no body tissue can squeeze through the gaps formed by the notches, and the compression bandage generates over the entire periphery a largely uniform compression pressure on the body extremity.

Advantageously included are fastening means of the fastening straps of the first and second group of the hook-and-loop closure, wherein the hook parts of the hook-and-loop closure are arranged on an outer end of each fastening strap of the first and second group, and the outside of the bandage body with loops corresponding to the hook parts, are designed for the adherence of the hook parts.

The bandage body and, advantageously, at least a subarea of the fastening strap are formed from a resilient material, in particular, from a short-stretch material with a stretching range between 15% and 50% of the unstretched state. The compression pressure exerted when the compression bandage is applied on the body extremity can thus advantageously be adjusted in the range between 8 mmHg and more than 40 mmHg, up to 60 mmHg, in that the bandage body is stretched due to pulling on the fastening straps, and the fastening straps are secured in a desired position on the outside of the bandage body.

In order to facilitate the application of the compression bandages on a body extremity, for example, a leg or an arm, the compression bandage has a pulling aid in the form of a tubular insert arranged on the inner side of the bandage body.

The tubular insert can here be formed from a tubular resilient textile material that is fastened, specifically, sewn, on the inner side of the bandage body. The pulling aid can also be formed from a cloth made of a resilient material fastened, specifically, sewn, on the inner side of the bandage body so that, between the inner side of the cloth and the inner side of the bandage body, a tubular gap forms, through which the body extremity to be treated can be guided when applying the compression bandage.

For the application of the compression bandage with the pulling aid, the body extremity to be treated is first passed through the tubular insert, and then the bandage body is wrapped in a tightly fitting manner around the body extremity. The fastening straps of the group of first fastening straps are subsequently pulled one after the other, for example, from proximal to distal, at first manually, so that they stretch the bandage body, and, thereafter, the first fastening straps are in each case fastened with their first fastening means onto the outside of the bandage body, wherein, for example, the hook parts at the end section of each fastening strap are placed on the outside of the bandage body and attached thereto. In this way, due to the pulling force exerted by the first fastening straps on the bandage body, a certain compression of the bandage body onto the body extremity inside is generated. If necessary, the fastening straps of the first group can be retightened by briefly loosening the fastening on the bandage body and reapplying it firmly at another site, where the respective fastening strap further stretches the bandage body.

In order to be able to further increase the compression pressure exerted by the fastening straps of the first group, the fastening straps of the first groups are also first tightened one after the other, for example, from proximal to distal, in order to further stretch the bandage body, and, thereafter, they are also fastened by means of the second fastening means on the outside of the bandage body, for example, by means of the hook parts of the hook-and-loop closures, which are arranged on the end areas of the fastening straps of the second group, and finally they are placed on the soft outside of the bandage body and attached there.

A particularly simple and rapid and fastening and loosening of the fastening straps is made possible when the first fastening means and/or the second fastening means comprise hook-and-loop closures, by means of which the fastening straps can be fastened to the outside of the bandage body, wherein the hook-and-loop closures here advantageously comprise hook parts which are fastened, in particular in the area of the free ends, to the fastening straps, and which work together with loops on the outside of the bandage body to establish a hook-and-loop closure connection. The hook-and-loop closures make it possible to fasten the free ends of the fastening straps to any site on the outside of the bandage body.

By means of the fastening straps of the first and second group, which, ultimately, in the applied state of the compression bandage, lie one on top of the other, it is possible to vary the compression pressure exerted by the compression bandage of the internally located body extremity and adjust it to a desired value to adapt it specifically and individually to the needs of the patient. By placing the fastening straps of the first and of the second group one on top of the other, the swollen body tissue is prevented from being able to pass through the gaps formed by notches in the bandage body, between adjacent fastening straps of the first group, since these gaps are covered by a fastening strap of the second group.

In order to prevent the compression bandage from being able to slip after it has been applied on the body extremity or from rolling up to form a bulge in case of movements of the body extremity, a stabilization element is arranged on the bandage body. The stabilization element can be formed, for example, by two, in particular arcuate, ribs sewn to the outside of the bandage body, which cross advantageously in the form of a V or an X. The V- or X-shaped crossing of the two ribs is advantageous particularly in compression bandages for the lower leg area, since the shape and the arrangement of the arcuate ribs with V- or X-shaped crossing can be anatomically adapted to the shape of the calf, so that the two ribs enclose the calf in the area of the V-shaped crossing or in the upper area of the X-shaped crossing. The arcuate ribs can be formed, for example, from a textile or a plastic material, which is sewn to the outside of the bandage body. In the process, the ribs stabilize the bandage body in the applied state of the compression bandage on the body extremity and prevent the compression bandage from being able to roll up to form a bulge, for example, starting from the hollow of the knee when the lower leg is at an angle.

For the anatomical conformation of the compression bandage to the shape of the body extremity against which the compression bandage is to be placed, it is advantageous if the bandage body has a curvature in a central area. Such a curvature can receive, for example, in a compression bandage for a lower leg, the convexly curved calf area of the lower leg, and as a result there is good fitting to the anatomy of the body extremity. In compression bandages provided for a body extremity with a joint (for example, an arm), a corresponding curvature in the central area of the bandage body can also be advantageous, in order to be able to accommodate the joint of the body extremity (for example, the arm joint), so that the mobility of the joint is not impaired by the applied compression bandage.

The production of the compression bandage is simplified if the bandage body is composed of two parts that are connected, specifically, sewn, to one another along a longitudinal seam. The curvature used for the fitting to the shape of the body extremity is arranged advantageously in the area of and symmetrically about the longitudinal seam. In order to stabilize the compression bandage on the body extremity, the curvature is preferably enclosed by two arcuate ribs sewn to the outside of the bandage body.

It is advantageous if the number of the fastening straps of the first group of fastening straps is greater by one than the number of the fastening straps of the second group. As a result, it is made possible that the notches, which separate the fastening straps of the first group from one another, are (completely) covered by the fastening strap of the second group, so that no body tissue can squeeze out through these notches, when the compression bandage is applied against the body extremity.

These and additional advantages and features of the disclosure result from the embodiment example described in further detail below in reference to the accompanying drawings. The drawings show:

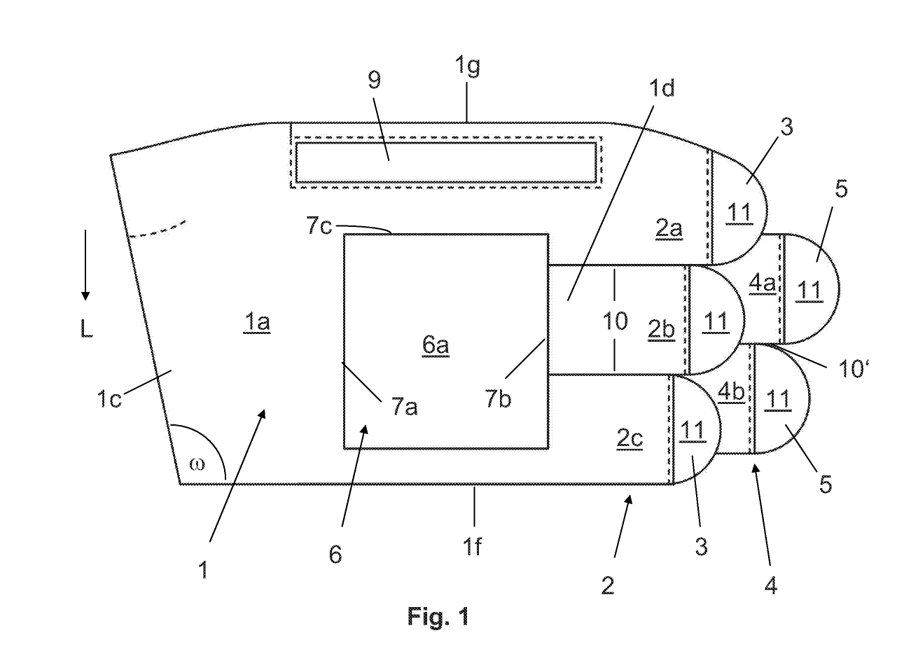

FIG. 1: Representation of the inner side of the compression bandage according to the disclosure;

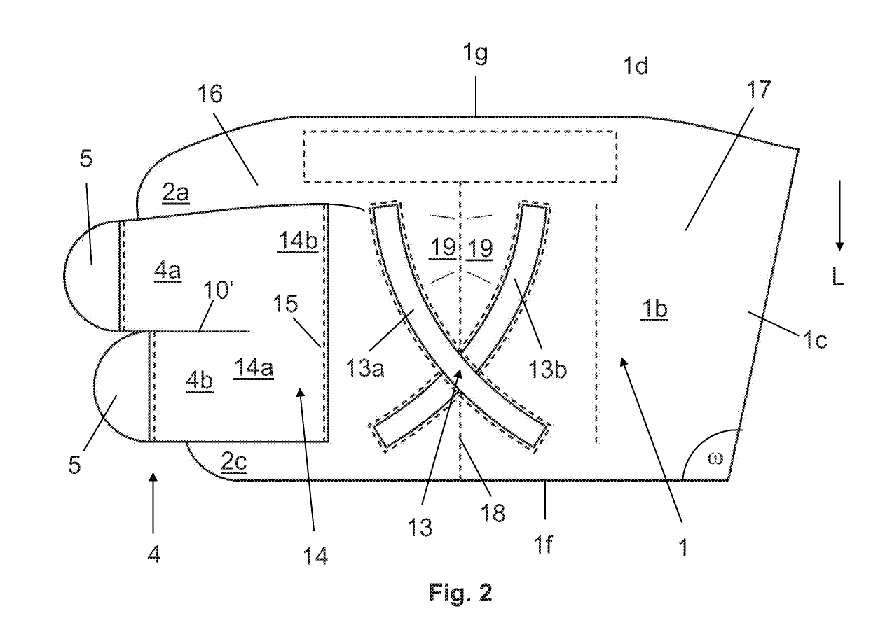

FIG. 2: Representation of the outside of the compression bandage of FIG. 1;

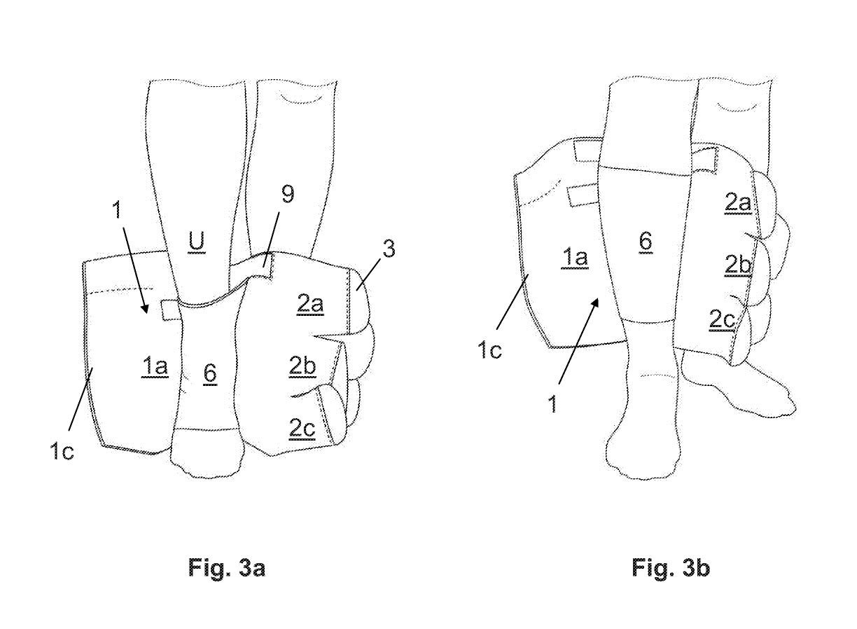

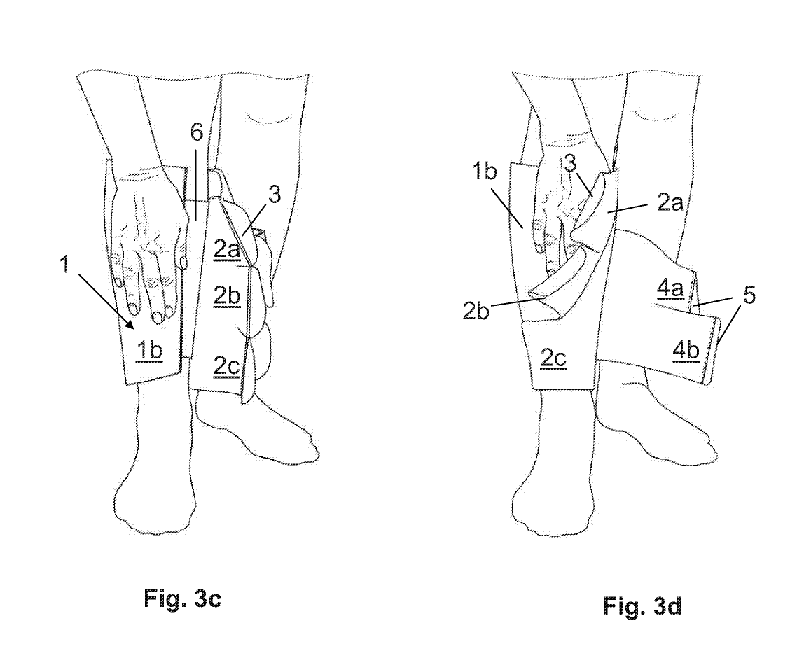

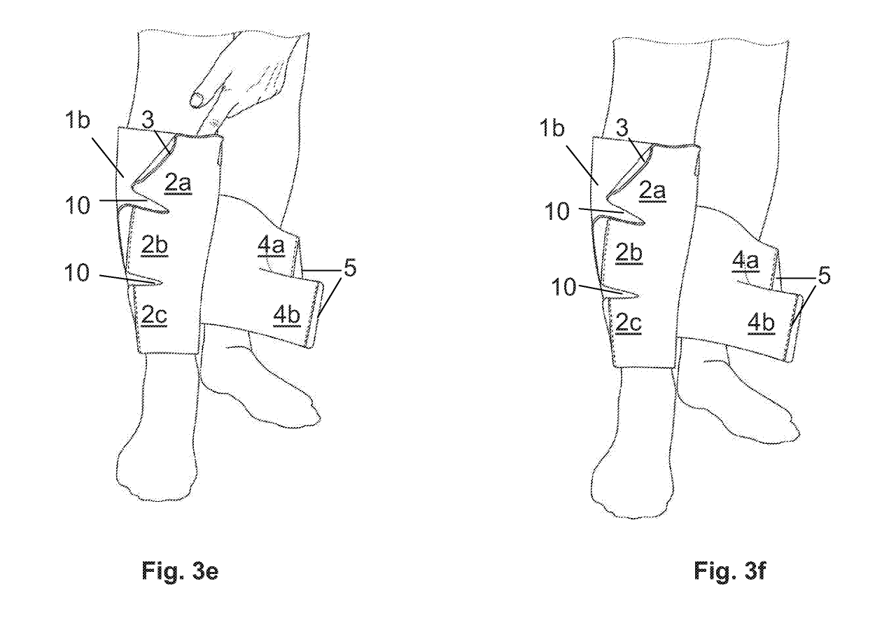

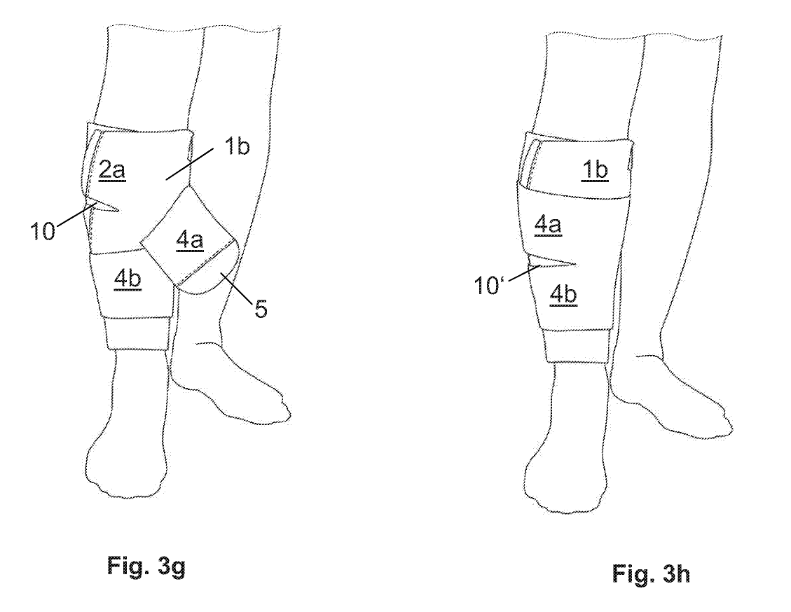

FIGS. 3a-3h: Representation of the steps for applying the compression bandage of FIGS. 1 and 2 to a lower leg of a patient.

The compression bandage shown in a top view in FIGS. 1 and 2 respectively comprises a bandage body 1 made of a resilient material. The resilient material can be, for example, a textile fabric made of polyamide and elastane, specifically, with 87% polyamide and 13% elastane, with respect to the weight of the textile fabric. The resilient material here has a material-specific stretching characteristic, which, advantageously allows a maximum stretching of the material in the range from 15 to 50% of the unstretched length. Preferably, the maximum stretching is either between 15 and 20% or between 25% and 35%.

The bandage body 1 has an inner side 1a, visible in FIG. 1, as well as an outside 1b, visible in FIG. 2. The bandage body 1 here comprises a first end section 1c and a second end section 1d as well as a distal edge if and a proximal edge 1g. The first end section 1c has a smooth edge extending in longitudinal direction L of the bandage body 1 (from distal to proximal), which advantageously encloses, with the distal edge 1f, an angle ω, which is greater than 90°, and which is, specifically, in the range from 100° to 120°. On the second end section 1d, a first group 2 of fastening straps is arranged. The fastening straps of the first group 2 here consist advantageously of the same resilient material as the bandage body 1. Advantageously, the fastening straps 2a, 2b, 2c of the first group 2 are formed by notches 10 in the second end section 1d of the bandage body 1 and separated from one another. The length of the notches 10 and thus the length of the fastening straps of the first group 2 is here preferably substantially smaller than the stretching of the bandage body 1 in the transverse direction (peripheral direction). Preferably, the length of the fastening straps 2a -2c of the first group 2 of fastening straps is between 2 cm and 5 cm.

In the embodiment of a compression bandage of the disclosure represented here in the drawing, the first group 2 of fastening straps comprises a total of three fastening straps 2a, 2b, 2c, which are arranged lying next to one another in longitudinal direction L of the bandage body 1, wherein adjacent fastening straps (2a, 2b; 2b, 2c) are separated from one another by a notch 10 in the bandage body. Due to the design of the fastening straps 2a, 2b, 2c of the first group 2, due to the notches 10 in the second end section 1d of the bandage body 1, adjacent fastening straps 2a, 2b; 2b, 2c lie directly next to one another, i.e., between the bottom edge of the upper (proximal) fastening strap 2a and the top edge of the distally adjoining strap 2b, there is only a small gap, which is formed by a notch 10. Correspondingly, between the upper edge of the lower (distal) fastening strap 2c and the lower edge of the fastening strap 2b adjoining it in proximal direction, there is only a small gap, which is also formed by a notch 10.

Each of the fastening straps 2a, 2b, 2c of the first group 2 of fastening straps comprises fastening means 3, by means of which the respective fastening strap 2a, 2b, 2c can be fastened to the outside 1b of the bandage body 1. Advantageously, the fastening means 3 are formed by hook-and-loop closures, wherein the hook parts of the hook-and-loop closures are arranged on the outer (free) ends of the fastening straps 2a, 2b, 2c, and the outside 1b of the fastening body 1 with loops is formed for the adherence of the hook parts 11 of the hook-and-loop closures. However, the fastening means 3 can also be formed by other means such as, for example, snap fasteners.

As shown in FIG. 2, on the outside 1b of the bandage body 1, a second group 4 of fastening straps is arranged. The second group 4 of fastening straps, in the embodiment example depicted here, comprises a compression bandage of the invention with two fastening straps 4a and 4b. The fastening straps 4a, 4b of the second group 4 here are arranged on a lashing strap 14 and extend from a first end section 14a of the lashing strap 14. The fastening straps 4a and 4b are separated from one another by a notch 10′ in the lashing strap 14. The lashing strap 14 and the fastening straps of the second groups 4 attached thereto are here advantageously made of the same resilient material as the bandage body 1. A second end section 14b of the lashing strap 14 has a smooth edge and is fastened to this edge on the outside lb of the bandage body 1, for example, by a seam 15.

The fastening straps 4a, 4b of the second group 4 of fastening straps are here arranged on the outside 1b of the bandage body 1, so that they cover the fastening straps 2a-2c of the first group 2 of fastening straps, in particular in the area of the notches 10 which separate adjacent fastening straps of the first group 2 from one another. Here, the fastening straps of the first group 2 are arranged offset with respect to one another, relative to the fastening straps of the second group 4 in longitudinal direction L of the bandage body 1 (from distal to proximal), so that, for example, a center section of the proximal fastening strap 4a of the second group 4 comes to rest above the notch 10 between the proximal fastening strap 2a of the first group and the fastening strap 2b adjoining it in distal direction, as can be seen in FIG. 1.

Like the fastening straps of the first group 2, the fastening straps of the second group 4 also each have second fastening means 5. Advantageously, the second fastening means 5 are also formed by hook-and-loop closures and comprise hook parts 11, which in each case are fastened to the free end of the fastening straps 4a, 4b of the second group 4 and arranged so that they can be connected to the outside 1b of the bandage body 1 for establishing a hook-and-loop closure connection.

The length of the fastening straps 4a, 4b of the second group 4 (that is, their extent transverse to the longitudinal direction L of the bandage body 1) is defined by the length of the notches 10′ and located advantageously in the area from ⅓ to ½ of the length of the lashing strap 14. Preferably, the length of the fastening straps 4a, 4b of the second group 4 is between 5 cm and 10 cm, and thus slightly longer than the length of the fastening straps of the first group 2.

On the outside 1b of the bandage body 1, as can be seen in FIG. 2, a stabilization element 13 is provided. The stabilization element 13, in the depicted embodiment example, is formed by two ribs 13a, 13b sewn to the outside 1b of the bandage body 1. The ribs 13a, 13b are here advantageously designed to be arcuate and arranged with respect to one another so that they form a V or—as shown in FIG. 1—an X. Here, the ribs 13a, 13b consist of a textile or plastic material, which is advantageously sewn to the outside of the bandage body 1. However, the ribs 13a, 13b can also fastened by bonding or fusing to the outside lb of the bandage body 1.

As also shown in FIG. 2, the bandage body 1 consists of two parts 16, 17, which are sewn to one another along a longitudinal seam 18. In the area of the longitudinal seam 18, at least in the proximal section, the bandage body 1 has a convexly shaped curvature 19 facing the outside 1b. The arcuate ribs 13a, 13b are here arranged on the outside 1a of the bandage body 1 in such a manner that they enclose the curvature 19. In the embodiment depicted in FIG. 2, the ribs 13a, 13b are arranged forming an X-shaped crossing with respect to one another and enclose the curvature 19 in the upper section of the “X”.

On the inner side 1a of the bandage body 1, a pulling aid in the form of a tubular insert 6 is arranged, as can be seen in FIG. 1. The tubular insert 6 is here formed by a resilient cloth 6a (textile material), which is fastened to the inner side 1a of the bandage body 1, specifically by sewing. The textile material of the tubular insert 6 is here designed to be substantially thinner than the resilient material of the bandage body 1, and it advantageously also consists of a mixture of polyamide and elastane, for example, with 71% polyamide and 29% elastane, with respect to the total weight of the resilient material of the tubular insert 6.

The resilient material of the bandage body 1 has a stretching characteristic with maximum stretching in the range from 10% to 50% of the unstretched state, and preferably in the range from 15% to 35% of the unstretched state.

In the embodiment example depicted in FIG. 1, the tubular insert 6 is formed by a seamless textile tube 6a, which is sewn along longitudinal seams 7a, 7b to the inner side 1a of the bandage body 1. The two longitudinal seams 7a, 7b here extend substantially parallel and at a distance from one another in the longitudinal direction L of the bandage body 1. Advantageously, the inner half of the textile tube 6b, facing the inner side 1a of the bandage body, is additionally sewn at its proximal edge along a peripheral seam 7c to the inner side 1a of the bandage body 1. On the inner side 1a of the bandage body 1, preferably between the proximal edge 1g and the peripheral seam 7c, an adhesive tape 9 is arranged, which extends in transverse direction (peripheral direction) of the bandage body 1.

In FIGS. 3a to 3h, the steps for applying the compression bandage on a body extremity of a patient are represented diagrammatically, where the body extremity is, for example, a lower leg U. For the application of the compression bandage on the lower leg U of the patient, the bandage body 1 is first wrapped around the lower leg U. For this purpose, the patient slips his or her lower leg U into the tubular insert 6 of the inner side 1a of the bandage body 1, until the calf comes to lie in the area of the curvature 19 of the bandage body 1 (FIGS. 3a and 3b). Then, the strap-free first end section 1c of the bandage body 1 is wrapped around half of the lower leg and held there (applied against the outside on the tubular insert 6). Then, the second end section 1d, on which the fastening straps of the first group 2 are arranged, is wrapped around the second half of the lower leg (FIG. 3c), and the fastening straps of the first group 2 are fastened by means of the first fastening means 3 to the outside 1b of the bandage body 1, as shown in FIG. 3d. In the process, due to the pull on the fastening straps 2 of the first group, a stretching of the bandage body 1 is achieved, which, due to the fastening of the fastening straps to the outside 1b of the bandage body 1, is fixed by means of the first fastening means 3. Advantageously, the fastening straps 2a-2c of the first group 2 are fastened after one another, for example, as shown in FIGS. 3d and 3e, from distal to proximal, on the outside 1b of the bandage body 1. After the fastening of the last fastening strap 2a of the first group 2, the fastening straps can be retightened, if need be, in that—advantageously again one after the other—they are briefly loosened from the bandage body 1 and, as a result of a pull applied at another site of the bandage body, fastened again, in order to exert a greater pull on the bandage body 1 at this other side (FIG. 3f). The pull exerted by the fastening straps of the first group 2 on the bandage body 1 produces a stretching of the resilient material of the bandage body 1, which exerts a compression on the lower leg.

In order to increase the compression exerted on the lower leg, the fastening straps of the second group 4 are also subsequently fastened to the outside lb of the bandage body 1. For this purpose, again, a pulling force is applied to each of the fastening straps of the second group 4, one after the other, for example, from proximal to distal, and said fastening straps are then also attached by means of the second fastening means 5 to the outside lb of the bandage body 1 (FIG. 3g). In the process, the fastening straps of the second group 4 are placed above the fastening straps of the first group 2, which are already fastened to the bandage body 1, so that the fastening straps of the second group 4 cover the fastening straps of the first group 2, as can be seen in FIGS. 3g and 3h. Due to the offset arrangement of the fastening straps of the second group 4, relative to the fastening straps of the first group 2, the notches 10 between adjacent fastening straps 2a, 2b; 2b, 2c of the first group 2 are, specifically, covered by the fastening straps of the second group 4. This prevents the swollen body tissue from being able to pass through in the area of the notches 10. As a result of the pull exerted by the fastening straps of the second group 4 on the bandage body 1, the bandage body 1 is stretched even further, to exert of an even higher compression on the lower leg. As a result of the site of the fastening of the fastening straps 4a, 4b of the second group 4 on the outside lb of the bandage body 1, the compression pressure exerted by the compression bandages on the lower leg U can be precisely dispensed and adjusted. The compression pressure exerted by the compression bandage results from the overall stretching of the bandage body 1, which is produced, on the one hand, by the pull of the fastening straps of the first group 2, and by the pull of the fastening straps of the second group 4.

The stabilization element 13 arranged on the outside lb of the bandage body 1 prevents a slipping of the applied compression bandage on the body extremity. Specifically, by means of the stabilization element 13, a bulge-shaped rolling up of the bandage body 1 is prevented. The curvature 19 in the central area of the bandage body 1 ensures an anatomical fitting of the bandage shape to the shape of the body extremity and thereby also prevents the ability of the applied compression bandage to slip. Moreover, the curvature 19 ensures a compression pressure evenly distributed over the periphery of the body extremity.

The disclosure is not limited to the embodiments depicted herein. Thus, for example, the number of the attachment straps of the first group of fastening straps or of the second group of fastening straps can be adapted to the size of the bandage. In the case of longer bandages, it is possible, for example, for the first group 2 to comprise four fastening straps and for the second group 4 to comprise three fastening straps. Here, it is advantageous if the number of the fastening straps of the first group 2 is greater by one than the number of the fastening straps of the second group 4.

The stretching characteristic of the resilient materials is variable over broad ranges and it can be adapted to the requirements of the compression treatment to be carried out with the bandage. Moreover, instead of hook-and-loop closures, other fastening means can also be used, such as, for example, snap fasteners or hook-and-eye connections. The embodiment example depicted shows a bandage for the right lower leg. For the use of the bandage with a left lower leg, the end sections 1c and 1d of the bandage body 1 are advantageously interchanged. This ensures that, when the bandage is applied, the fastening means 3, 5 of the first and second fastening straps 2, 4 in each case can be fastened laterally to the outside of the bandage body 1. The compression bandage of the disclosure can, furthermore, also be applied on other body extremities, for example, on an upper leg or a forearm or upper arm.

What is claimed is:

1. A compression bandage for treating lymphatic edema by applying the compression bandage on a body extremity, wherein the compression bandage comprises:
a bandage body made of a resilient material to be wrapped around the body extremity, with an inner side and an outside, as well as with a first and a second end section,
a first group of fastening straps, which are arranged on an end section of the bandage body, wherein the fastening straps of the first group are separated from one another by notches in the bandage body and, in each case, have first fastening elements, by which the respective fastening strap of the first group can be fastened to the outside of the bandage body, when the body bandage is wrapped around the body extremity,
wherein a second group of fastening straps which are fastened to the outside of the bandage body and, when the compression body is applied against the body extremity, are arranged so that they cover the notches which separate the fastening straps of the first group from one another.

2. The compression bandage according to claim 1, wherein the fastening straps of the second group each have second fastening elements, by which the respective fastening strap of the second group can be fastened to the outside of the bandage body.

3. The compression bandage according to claim 1, wherein the compression bandage exerts a compression pressure on the body extremity, when the bandage body is wrapped around the body extremity, and the fastening straps of the first and second group are fastened to the outside of the bandage body, wherein the compression pressure exerted by the compression bandage on the body extremity can be adjusted by positioning the fastening of the fastening straps on the outside of the bandage body.

4. The compression bandage according to claim 1, wherein the first fastening elements and/or the second fastening elements comprise hook-and-loop closures and hook closures or snap fasteners.

5. The compression bandage according to claim 1, wherein the fastening straps of the first group are arranged on one of the two end sections of the bandage body, and in that the other end section comprises a strap-free outer edge.

6. The compression bandage according to claim 1, wherein the first group of fastening straps comprises three or four fastening straps, and wherein the second group of fastening straps comprises two or three fastening straps.

7. The compression bandage according to claim 1, wherein the fastening tubes of the first group of fastening straps are arranged lying next to one another in the longitudinal direction of the bandage body, and wherein the fastening straps of the second group of fastening straps are arranged lying next to one another in longitudinal direction of the bandage body.

8. The compression bandage according to claim 1, wherein the fastening straps of the second group are arranged offset in the longitudinal direction of the bandage body with respect to the fastening straps of the first group.

9. The compression bandage according to claim 1, wherein the fastening straps of the second group are arranged on a lashing strap and extend from a first end section of the lashing strap.

10. The compression bandage according to claim 9, wherein a second end section of the lashing strap is fastened, specifically, sewn, on the outside of the bandage body.

11. The compression bandage according to claim 1, wherein on the inner side of the bandage body, a pulling strap in the form of a tubular insert is arranged, wherein the tubular insert is formed from a resilient textile material and fastened, specifically, sewn, on the inner side of the bandage body.

12. The compression bandage according to claim 11, wherein the tubular insert is made from a resilient textile material and fastened, specifically, sewn, on the inner side of the bandage body.

13. The compression bandage according to claim 11, wherein half of the tubular insert is formed by the inner side of the bandage body and the other half is formed by a cloth fastened to the inner side of the bandage body.

14. The compression bandage according to claim 11, wherein the tubular insert is formed by a textile tube, which is fastened to the inner side of the bandage body along a first and a second longitudinal seam and/or along at least one peripheral seam.

15. The compression bandage according to claim 1, wherein on the outside of the bandage body, a stabilization element is arranged.

16. The compression bandage according to claim 15, wherein the stabilization element is formed by two, in particular arcuate, ribs sewn on the outside of the bandage body.

17. Compression bandage according to claim 16, characterized in that the ribs cross in the form of a V or X.

18. The compression bandage according to claim 1, wherein the bandage body is composed of two parts, which are connected, specifically, sewn, to one another along a longitudinal seam.

19. The compression bandage according to claim 1, wherein in a central area, in particular in the area of the longitudinal groove, the bandage body has at least in some sections a shaped curvature facing the outside.

20. The compression bandage according to claim 19, wherein the curvature is enclosed by two arcuate ribs sewn on the outside of the bandage body.
